(19) United States
(12) Patent Application Publication
(10) Pub. No.: US 2026/0152525 A1
(43) Pub. Date: Jun. 4, 2026

(54) METHOD FOR SYNTHESIZING PEPTIDE

(71) Applicant: SEKISUI MEDICAL CO., LTD., Tokyo (JP)

(72) Inventor: Shinya YANO, Chuo-ku (JP)

(73) Assignee: SEKISUI MEDICAL CO., LTD., Tokyo (JP)

(21) Appl. No.: 19/125,358

(22) PCT Filed: Nov. 9, 2023
(86) PCT No.: PCT/JP2023/040451

§ 371 Date: Apr. 29, 2025

(30) Foreign Application Priority Data
Nov. 10, 2022 (JP) 2022-180283

(51) Int. Cl. C07K 1/06; C07K 1/02; C07K 1/04

(52) U.S. Cl. CPC C07K 1/061; C07K 1/02; C07K 1/042

(57) ABSTRACT

To provide synthesis method where efficiencies of Fmoc amino acid condensation reaction and deFmoc reaction are not impaired without additional solvent, in synthesis of peptide by using certain number of hydrophobic amino acids and amino acid having functional group in its side chain or long-chain peptide.

Method for synthesizing peptide comprising one or more amino acid having functional group in its side chain, 50% or more of hydrophobic amino acid, and 10% or less of amino acid wherein the α-amino group is secondary amino group; or peptide comprising one or more amino acid having functional group in its side chain and having 21 or more of amino acid residues, wherein the carrier for peptide synthesis of Formula (1) is introduced into the side chain of the amino acid having functional group in its side chain.

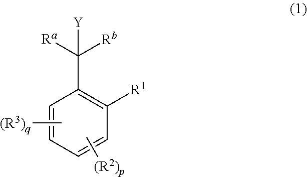

### TECHNICAL FIELD

The present invention relates to a method for synthesizing a peptide.

### BACKGROUND ART

In the method for synthesizing a peptide, Fmoc, Boc, Cbz, Ac, and the like are used as protecting groups of an amino group. Among them, Fmoc is widely used because it imparts lipid solubility to the protected compound, it is stable in a neutral to acidic region, and it can be easily eliminated by using an amine compound.

When a peptide sequence to be synthesized contains a certain number of hydrophobic amino acids and the number of amino acids in which an α-amino group is a secondary amino group is 10% or less, or when a peptide sequence to be synthesized has as long chain as 21 residues or more, the efficiency of the subsequent Fmoc amino acid condensation reaction and the efficiency of the deFmoc reaction may be impaired. In particular, in liquid-phase peptide synthesis, it is sometimes impossible to conduct liquid separation due to gelation of the reaction liquid, which interrupted from proceeding to the subsequent condensation step.

In view of this, by adding an additional solvent to the reaction liquid for peptide synthesis to reduce the peptide concentration in the reaction liquid, synthesis of a peptide sequence that is likely to gel has been realized. However, an increase in the amount of the reaction liquid has been a disadvantage in mass synthesis and purification.

### CITATION LIST

### Patent Literature

Patent Literature 1: Japanese Patent No. 6116782B
Patent Literature 2: Japanese Patent No. 6201076B
Patent Literature 3: Japanese Patent No. 6283775B
Patent Literature 4: Japanese Patent No. 6322350B
Patent Literature 5: Japanese Patent No. 6393857B
Patent Literature 6: Japanese Patent No. 6531235B

### SUMMARY OF INVENTION

### Technical Problem

The present invention aims to provide a synthesis method in which, in peptide synthesis using amino acids containing a certain number of hydrophobic amino acids and an amino acid having a functional group in the side chain, the efficiency of a condensation reaction of Fmoc amino acids or the deFmoc reaction efficiency is not impaired without adding an additional solvent.

### Solution to Problem

Accordingly, the present inventors made energetic investigations in order to solve the above problems, and found that when peptide synthesis is performed by protecting a functional group of an amino acid having a functional group in the side chain with a carrier for peptide synthesis having a silyloxy group represented by the following general Formula (1), problems such as gelation of a reaction liquid do not occur even without adding an additional solvent, the efficiency of a condensation reaction of Fmoc amino acids or the deFmoc reaction efficiency is maintained high, and peptide synthesis can be efficiently performed, thereby completing the present invention.

That is, the present invention provides the following to [7].
[1] A method for synthesizing a peptide comprising one or more amino acid having a functional group in a side chain thereof, 50% or more of hydrophobic amino acid, and 10% or less of amino acid in which an α-amino group is a secondary amino group; or a peptide comprising one or more amino acid having a functional group in a side chain thereof, and 21 or more of amino acid residues,
   wherein a carrier for peptide synthesis represented by a general Formula (1) below is introduced into the side chain of the amino acid having a functional group in a side chain thereof:

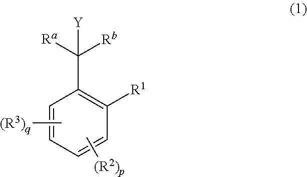

wherein,
R¹ is a hydrogen atom; or when R^{b} is a group represented by Formula (a) below, R¹ may be a fluorene ring taken together with Formula (a), a xanthene ring taken together with Formula (a) via an oxygen atom, or a 9,10-dihydroanthracene ring taken together with Formula (a) via a methylene group;
p R²'s each independently represent a group represented by Formula (2);

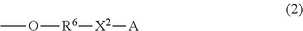

R⁶ represents a linear or branched alkylene group having 1 to 16 carbon atoms;
X² represents O or CONR¹⁷ (wherein R¹⁷ represents a hydrogen atom or an alkyl group having 1 to 4 carbon atoms); and
A represents a group represented by Formula (3), (4), (5), (6), (7), (8), (9), (10), (11), (12) or (13),

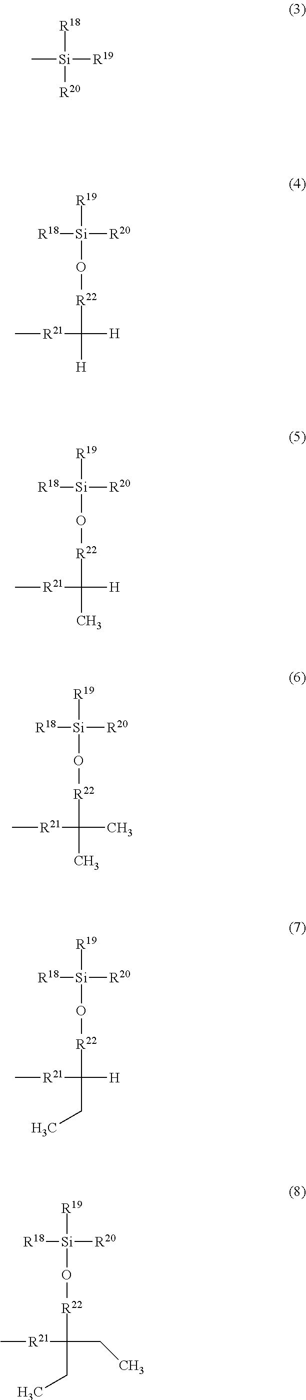

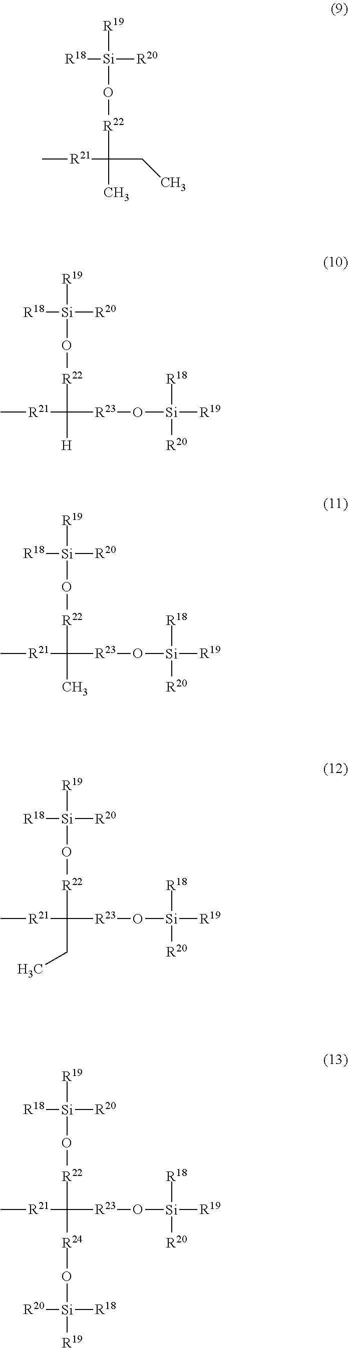

wherein, R¹⁸, R¹⁹, and R²⁰ are the same or different and each represent a linear or branched alkyl group having 1 to 6 carbon atoms or an aryl group optionally having a substituent; and R²¹ represents a single bond or a linear or branched alkylene group having 1 to 3 carbon atoms, and R²², R²³, and R²⁴ each represent a linear or branched alkylene group having 1 to 3 carbon atoms,
q R³'s each independently represent a hydrogen atom, a halogen atom, an alkyl group having 1 to 4 carbon atoms, or an alkoxy group having 1 to 4 carbon atoms;
p represents an integer of 1 to 4, and q represents an integer of 0 to 3; and
R^{a} and R^{b} each independently represent a hydrogen atom, a linear or branched alkyl group having 1 to 4 carbon atoms, an aralkyl group, or a group represented by Formula (a);

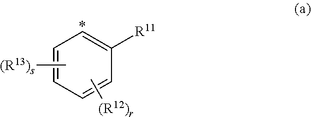

wherein, * represents a bonding site;
R¹¹ may be a hydrogen atom or may form a fluorene ring taken together with R¹ in Formula (1), a xanthene ring taken together with R¹ in Formula (1) via an oxygen atom, or a 9,10-dihydroanthracene ring taken together with R¹ in Formula (1) via a methylene group;
r R¹²'s each independently represent a group represented by Formula (2) above (R⁶, X², and A in Formula (2) are the same as described above);
s R¹³'s each independently represent a hydrogen atom, a halogen atom, an alkyl group having 1 to 4 carbon atoms, or an alkoxy group having 1 to 4 carbon atoms; and
s represents an integer of 1 to 4 and r represents an integer of 0 to 3,
Y represents a hydroxy group, NHR²⁵ (R²⁵ represents a hydrogen atom, an alkyl group, or an aralkyl group), or a halogen atom.
[2] The method for synthesizing a peptide according to [1], in which the hydrophobic amino acid is selected from the group consisting of glycine, alanine, valine, leucine, isoleucine, methionine, phenylalanine, and tryptophan.
[3] The method for synthesizing a peptide according to or [2], wherein a number of amino acid in which the carrier for peptide synthesis represented by the general Formula (1) is introduced into the side chain is 1 to 4 residues.
[4] The method for synthesizing a peptide according to any one of [1] to [3], wherein the amino acid having a functional group in a side chain thereof is an amino acid in which the carrier for peptide synthesis represented by the general Formula (1) is previously introduced into the side chain.
[5] The method for synthesizing a peptide according to any one of [1] to [3], wherein the carrier for peptide synthesis represented by the general Formula (1) is introduced after an amino acid side chain contained in a peptide is deprotected.
[6] The method for synthesizing a peptide according to any one of [1] to [5], wherein the amino acid in which the carrier for peptide synthesis represented by the general Formula (1) is introduced into the side chain is selected from the group consisting of aspartic acid, glutamic acid, and cysteine.
[7] The method for synthesizing a peptide according to any one of [1] to [6], which is a liquid-phase synthesis method.

### Advantageous Effects of Invention

The present invention facilitates to provide a synthesis method in which, in synthesis of a peptide by using amino acids containing a certain number of hydrophobic amino acids and an amino acid having a functional group in a side chain thereof, or a long-chain peptide, the efficiency of a condensation reaction of Fmoc amino acids and the efficiency of a deFmoc reaction are not impaired without adding an additional solvent.

### BRIEF DESCRIPTION OF DRAWINGS

FIG. 1 is a view showing a state of liquid separation of a reaction liquid of Example (1-e).

FIG. 2 is a view showing a state of liquid separation of a reaction liquid of Comparative Example (1-e).

FIG. 3 is a view showing a state of liquid separation of a reaction liquid of Example (2-h).

FIG. 4 is a view showing a state of liquid separation of a reaction liquid of Comparative Example (2-b).

### DESCRIPTION OF EMBODIMENTS

An aspect of a method for synthesizing a peptide of the present invention is a method for synthesizing a peptide containing one or more amino acids having a functional group in a side chain, containing 50% or more of hydrophobic amino acids, and containing 10% or less of amino acids in which an α amino group is a secondary amino group, or a peptide containing one or more amino acids having a functional group in a side chain, and containing 21 amino acid residues or more, in which a carrier for peptide synthesis represented by a general Formula (1) below is introduced into a side chain of an amino acid having a functional group in a side chain.

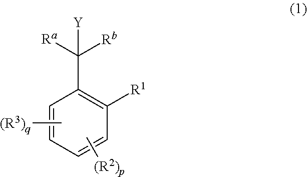

In the formula,
R¹ is a hydrogen atom; or when R^{b} is a group represented by Formula (a) below, R¹ is a fluorene ring taken together with Formula (a), a xanthene ring taken together with Formula (a) via an oxygen atom, or a 9,10-dihydroanthracene ring taken together with Formula (a) via a methylene group;
p R²'s each independently represent a group represented by Formula (2);

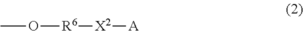

R⁶ represents a linear or branched alkylene group having 1 to 16 carbon atoms;
X² represents O or CONR¹⁷ (here, R¹⁷ represents a hydrogen atom or an alkyl group having 1 to 4 carbon atoms); and
A represents a group represented by Formula (3), (4), (5), (6), (7), (8), (9), (10), (11), (12) or (13),

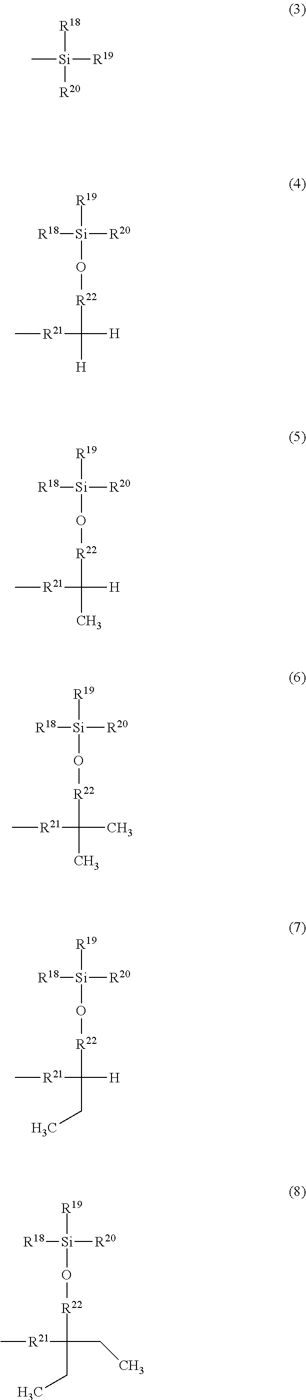

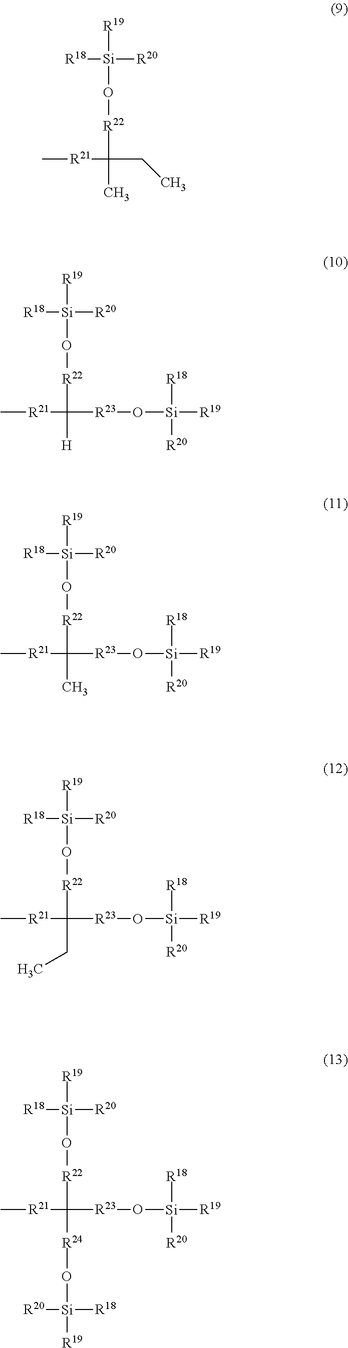

wherein, R¹⁸, R¹⁹, and R²⁰ are the same or different and each represent a linear or branched alkyl group having 1 to 6 carbon atoms or an aryl group optionally having a substituent; and R²¹ represents a single bond or a linear or branched alkylene group having 1 to 3 carbon atoms, and R²², R²³, and R²⁴ each represent a linear or branched alkylene group having 1 to 3 carbon atoms,
q R³'s each independently represent a hydrogen atom, a halogen atom, an alkyl group having 1 to 4 carbon atoms, or an alkoxy group having 1 to 4 carbon atoms;
p represents an integer of 1 to 4, and q represents an integer of 0 to 3; and
R^{a} and R^{b} each independently represent a hydrogen atom, a linear or branched alkyl group having 1 to 4 carbon atoms, an aralkyl group, or a group represented by Formula (a);

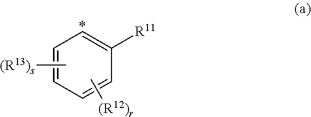

wherein, * represents a bonding site;
R¹¹ may be a hydrogen atom or may form a fluorene ring taken together with R¹ in Formula (1), a xanthene ring taken together with R¹ in Formula (1) via an oxygen atom, or a 9,10-dihydroanthracene ring taken together with R¹ in Formula (1) via a methylene group;
r R¹²'s each independently represent a group represented by Formula (2) above (R⁶, X², and A in Formula (2) are the same as described above);
s R¹³'s each independently represent a hydrogen atom, a halogen atom, an alkyl group having 1 to 4 carbon atoms, or an alkoxy group having 1 to 4 carbon atoms; and
s represents an integer of 1 to 4 and r represents an integer of 0 to 3,
Y represents a hydroxy group, NHR²⁵ (R²⁵ represents a hydrogen atom, an alkyl group, or an aralkyl group), or a halogen atom.

In the constituent amino acid of the peptide obtained by the method of the present invention, (a) one or more amino acids having a functional group is contained in the side chain, the proportion of hydrophobic amino acids is 50% or more, and the proportion of amino acids in which the α-amino group is a secondary amino group is 10% or less, or (b) one or more amino acids having a functional group in the side chain, and the number of amino acid residues of the peptide is 21 or more. Other constituent amino acids can be randomly selected as long as the peptide satisfies the above conditions.

In the synthesis of such a peptide containing a large amount of hydrophobic amino acids or long-chain peptide, as described above, the efficiency of the condensation reaction of Fmoc amino acids or the deFmoc reaction efficiency is impaired. The present invention facilitates to prevent a decrease in the efficiency of the Fmoc amino acid condensation reaction or the deFmoc reaction efficiency.

In the peptide synthesis of the present invention, one or more amino acids having a functional group in the side chain are used. It is preferable to introduce the carrier for peptide synthesis represented by the general Formula (1) into the side chain functional group of the amino acid from the viewpoint of not reducing the efficiency of the condensation reaction of the Fmoc amino acid or the deFmoc reaction efficiency without adding an additional solvent.

The number of amino acid in which the carrier for peptide synthesis represented by the general Formula (1) is introduced into the side chain may be 1 or more, and is more preferably 1 to 4 residues.

In addition, an amino acid in which the carrier for peptide synthesis represented by the general Formula (1) is previously introduced into the amino acid side chain may be used, or the carrier for peptide synthesis represented by the general Formula (1) may be introduced by deprotecting the side chain of the amino acid in which the side chain contained in the peptide is protected.

The amino acid in which the carrier for peptide synthesis represented by the general Formula (1) is introduced into the side chain may be an amino acid having a functional group in the side chain, and an amino acid having a carboxyl group or a mercapto group in the side chain is preferable, and aspartic acid, glutamic acid, or cysteine is more preferable.

In the peptide synthesis of the present invention, the proportion of hydrophobic amino acids in the constituent amino acids of the peptide to be synthesized is 50% or more, and more preferably 60% or more. In addition, the upper limit of the proportion of the hydrophobic amino acid among the constituent amino acids of the peptide is preferably 95% or less, and more preferably 90% or less.

The hydrophobic amino acid is an amino acid having a non-polar property, and specific examples thereof include an amino acid having a hydrocarbon group in the side chain, and include glycine, alanine, valine, leucine, isoleucine, methionine, phenylalanine, and tryptophan.

In the peptide synthesis of the present invention, the proportion of amino acids in which the α amino group is a secondary amino group among constituent amino acids of the peptide to be synthesized is preferably 10% or less. Here, 10% or less includes 0%.

Examples of the amino acid in which the α amino group is a secondary amino group include proline, proline derivatives, pseudoproline, pseudoproline derivatives, N-methylamino acids, and N-methylamino acid derivatives.

In the peptide synthesis of the present invention, the peptide to be synthesized is composed of two or more amino acids, preferably three or more amino acids, and more preferably four or more amino acids.

In the synthesis of the long-chain peptide of the present invention (peptide having 21 amino acid residues or more), the constituent amino acids include one or more amino acids having a functional group in the side chain. Here, as the amino acid having a functional group in the side chain, the same amino acid as described above is used in the same amount as described above. The number of hydrophobic amino acids or the proportion of amino acids in which the α-amino group is a secondary amino group are not limited.

The target peptide may have 21 amino acid residues or more, and may be a peptide having several tens of amino acid residues.

Next, the carrier for peptide synthesis represented by the general Formula (1) will be described. The carrier for peptide synthesis represented by the general Formula (1) is a compound described in Patent Literature 1 to 6, and is known to be useful as a protecting group for peptides and amino acids. However, it is not known whether this is applicable to the problem of the present invention, in the synthesis of a peptide using a large amount of hydrophobic amino acids or a long-chain peptide.

Formula (1) includes the following five types of skeletons (A) to (E) depending on the types of R^{a}, R^{b}, R¹, and R¹¹.

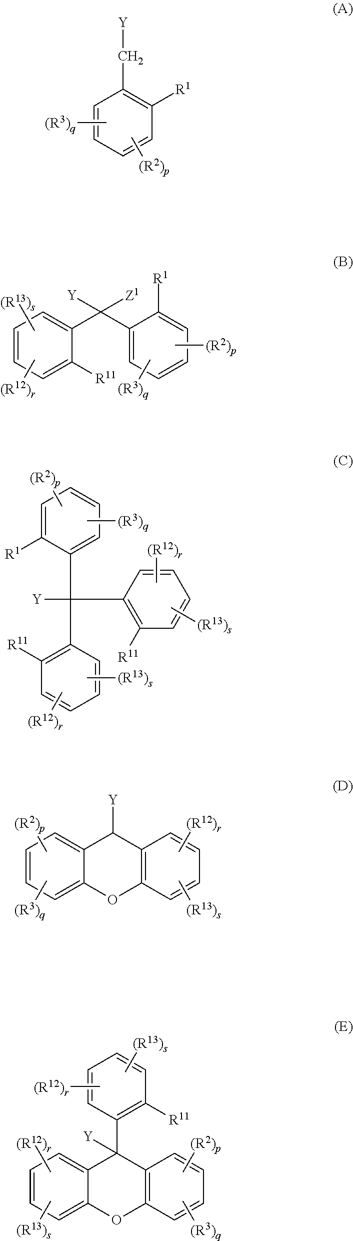

In the formula, Z¹ represents a hydrogen atom, a linear or branched alkyl group having 1 to 4 carbon atoms, or an aralkyl group, Z² represents an oxygen atom, a single bond, or a methylene group, and other symbols represent the same meaning as described above.

In the above formula, Y represents a hydroxy group, NHR²⁵ (R²⁵ represents a hydrogen atom, an alkyl group, or an aralkyl group), or a halogen atom. Among them, a hydroxy group, an amino group, or a halogen atom is preferable.

The alkyl group is preferably a linear or branched alkyl group having 1 to 4 carbon atoms, and more preferably a methyl group, an ethyl group, an isopropyl group, or the like. The aralkyl group is preferably a phenyl-Cl-4 alkyl group or a diphenyl-Cl-4 alkyl group, and more preferably a benzyl group, a phenethyl group, or a diphenylmethyl group. The halogen atom is preferably a chlorine atom, a bromine atom, or an iodine atom.

R¹ and R¹¹ preferably represent a hydrogen atom in the skeletons (A) to (E).

Among the p R²'s and the r R¹²'s, 1 to 4 of each represent a group represented by Formula (2), and 2 to 4 of each are preferably a group represented by Formula (2).

R⁶ represents a linear or branched alkylene group having 1 to 16 carbon atoms. The number of carbon atoms in the alkylene group is preferably 2 or more, more preferably 6 or more, and still more preferably 8 or more, and is preferably 16 or less, more preferably 14 or less, and still more preferably 12 or less.

Among the alkylene groups, a linear or branched alkylene group having 2 to 16 carbon atoms is preferable, a linear or branched alkylene group having 6 to 16 carbon atoms is more preferable, a linear or branched alkylene group having 8 to 14 carbon atoms is still more preferable, and a linear or branched alkylene group having 8 to 12 carbon atoms is still more preferable. Specific examples of the alkylene group include a methylene group, an ethylene group, a trimethylene group, a tetramethylene group, a pentamethylene group, a hexamethylene group, a heptamethylene group, an octamethylene group, a nanomethylene group, a decamethylene group, an undecamethylene group, a dodecamethylene group, and a tetradecamethylene group.

X² represents O or CONR¹⁷.

Here, R¹⁷ represents a hydrogen atom or an alkyl group having 1 to 4 carbon atoms, and is preferably a hydrogen atom.

A represents a group represented by Formula (3), (4), (5), (6), (7), (8), (9), (10), (11), (12) or (13). R¹⁰, R¹⁰, and R²⁰ are the same or different and each represent a linear or branched alkyl group having 1 to 6 carbon atoms or an aryl group optionally having a substituent. Here, examples of the alkyl group having 1 to 6 carbon atoms include a methyl group, an ethyl group, a n-propyl group, an isopropyl group, a n-butyl group, an isobutyl group, a sec-butyl group, a tert-butyl group, a n-pentyl group, and a n-hexyl group. Among them, an alkyl group having 1 to 4 carbon atoms is more preferable, and a methyl group, tert-butyl, and isopropyl group are still more preferable.

Examples of the aryl group optionally having a substituent include an aryl group having 6 to 10 carbon atoms, and specifically include a phenyl group and a naphthyl group which may be substituted by an alkyl group having 1 to 3 carbon atoms. Among them, a phenyl group is more preferable.

R²¹ represents a single bond or a linear or branched alkylene group having 1 to 3 carbon atoms. Examples of the linear or branched alkylene group having 1 to 3 carbon atoms include a methylene group, an ethylene group, a trimethylene group, and a propylene group, and among these, a single bond is particularly preferable.

R²², R²³, and R²⁴ each represent a linear or branched alkylene group having 1 to 3 carbon atoms. Examples of the linear or branched alkylene group having 1 to 3 carbon atoms include a methylene group, an ethylene group, a trimethylene group, and a propylene group, and a methylene group is particularly preferable.

q R³'s or s R¹³'s each independently represent a hydrogen atom, a halogen atom, an alkyl group having 1 to 4 carbon atoms, or an alkoxy group having 1 to 4 carbon atoms. Examples of the halogen atom include a fluorine atom, a chlorine atom, a bromine atom, and an iodine atom. Examples of the alkyl group having 1 to 4 carbon atoms include a methyl group, an ethyl group, an isopropyl group, an n-propyl group, and an n-butyl group. Examples of the alkoxy group having 1 to 4 carbon atoms include a methoxy group, an ethoxy group, and an isopropyloxy group. q and s are each preferably 0 to 3.

The method for synthesizing a peptide of the present invention can be carried out in accordance with an ordinary peptide synthesis method except that the carrier for peptide synthesis represented by the general Formula (1) is introduced into the side chain of an amino acid having a functional group in the side chain. For example, included is a method in which an amino acid or a peptide obtained by introducing a carrier for peptide synthesis represented by the general Formula (1) into the side chain functional group and a carboxyl group and an amino acid in which an amino group is protected are condensed, then a protecting group of the amino group is eliminated, and the next amino acid is reacted in the same manner. Here, examples of the protecting group of the amino group include an Fmoc group, a Boc group, a Cbz group, an Ac group, and the like, and it is preferable to use an Fmoc group.

In addition, the method for synthesizing a peptide of the present invention may be a solid phase method or a liquid-phase method, but is preferably performed by a liquid-phase method capable of mass production. To perform the method for synthesizing a peptide of the present invention by the solid-phase method, for example, an insoluble resin carrier can be bound to the C-terminal side of the peptide chain. To perform the method for synthesizing a peptide of the present invention by the liquid-phase method, in the peptide chain elongation step, the peptide chain being synthesized may contain another carrier for liquid-phase peptide synthesis in addition to the carrier for peptide synthesis introduced into the side chain functional group. The other carrier for liquid-phase peptide synthesis may be the same as or different from the carrier for peptide synthesis introduced into the side chain functional group, and can be introduced, for example, into the C-terminal side of the peptide chain. The method for synthesizing a peptide of the present invention in the liquid-phase method is preferably performed through, for example, the following steps.
a. condensing an amino acid, a peptide or an amino acid amide obtained by introducing the carrier for peptide synthesis into a side chain functional group with an amino acid or a peptide in which an amino group is protected by an Fmoc group in a solvent containing an organic solvent;
b. adding an amino acid active ester scavenger to a reaction liquid after a condensation reaction;
c. eliminating the Fmoc group of a compound in which an amino group is protected by the Fmoc group in the reaction liquid; and
d. adding an aqueous solution which may contain a base or the like to the reaction liquid and then separating the solution to afford an organic solvent layer containing a condensate of the amino acid, the peptide or the amino acid amide in which the carrier for peptide synthesis is introduced and the amino acid or the peptide from which the Fmoc group is eliminated.

Here, instead of the a., the following process may be performed.
e. condensing an amino acid, a peptide, or an amino acid amide with an amino acid or a peptide, in which an amino group is protected by an Fmoc group and a side chain functional group is protected, in a solvent containing an organic solvent, and
f. deprotecting a part or all of a side chain protecting group of an amino acid in which the side chain functional group condensed is protected in e., or a side chain protecting group of a peptide in which the side chain functional group is protected condensed in e., and introducing a carrier for peptide synthesis represented by the general Formula (1).

In passing via the steps e. and f., it is preferable that a protecting group other than the protecting group of the amino acid side chain into which the carrier for peptide synthesis is introduced is not deprotected in the step of deprotecting the side chain functional group.

According to the method of the present invention, problems such as gelation of the reaction liquid do not occur even when an additional solvent is not added, the efficiency of the condensation reaction of Fmoc amino acids or the deFmoc reaction efficiency is maintained high, and peptide synthesis can be efficiently performed.

### EXAMPLES

Next, the present invention will be described in more detail with reference to Examples, but the present invention is not limited to these Examples.

### Reference Example 1

### Synthesis of Fmoc-Asp(B2-STag)-OH

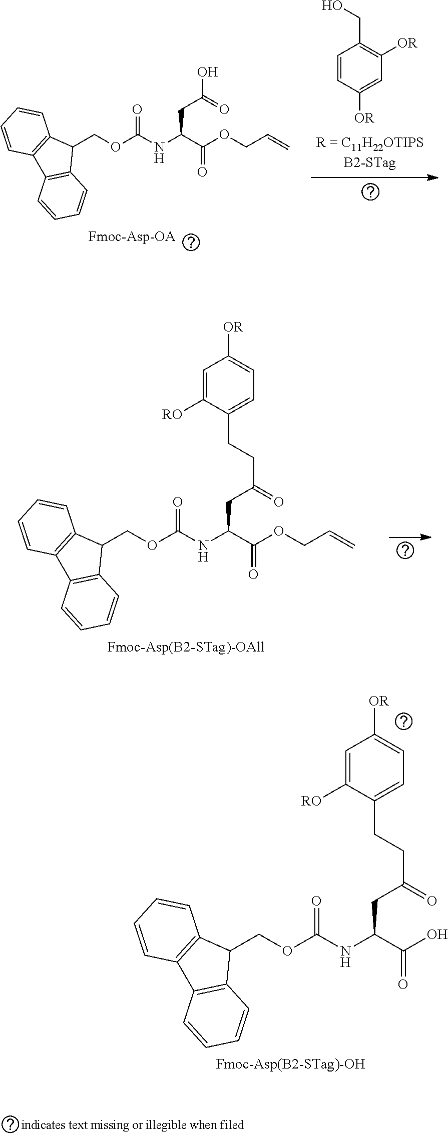

Fmoc-Asp-OAll, B2-STag, Fmoc-Asp(B2-STag)-OAll, and Fmoc-Asp(B2-STag)-OH represent the structure in the formula. The following amino acids represent L-forms.

### Reference Example (1-a)

3.00 g (3.78 mmol) of B2-STag (manufactured by SEKISUI MEDICAL CO., LTD.) and 4.49 g (11.3 mmol) of Fmoc-Asp-OAll were dissolved in a solution mixture of 26.5 mL of tetrahydrofuran (THF) and 11.3 mL of N,N-dimethylformamide (DMF), and added with 2.17 g (11.3 mmol) of 1-(3-dimethylaminopropyl)-3-ethylcarbodiimide hydrochloride (EDCI·HCl) and 92.4 mg (0.756 mmol) of 4-dimethylaminopyridine (DMAP), and the mixture were stirred at room temperature for 40 minutes. The reaction solution was concentrated under reduced pressure, and the obtained residue was washed by liquid-liquid extraction with 177 mL of heptane and 177 mL of acetonitrile (MeCN). The obtained heptane layer was washed by liquid-liquid extraction with 11.1 mL of heptane and 177 mL of MeCN. The washing by liquid-liquid extraction with heptane and MeCN was further performed twice, and the heptane layer was concentrated and dried under reduced pressure to afford 4.16 g of Fmoc-Asp(B2-STag)-OAll.

ESIMS (m/z) 1188.0 (M+NH₄)⁺

### Reference Example (1-b)

2.17 g (1.85 mmol) of Fmoc-Asp(B2-STag)-OH was dissolved in 18.5 mL of anhydrous THF, 0.107 g (0.0927 mmol) of tetrakis(triphenylphosphine)palladium (0) (Pd (Ph₃)₄) and 0.455 mL (3.71 mmol) of phenylsilane were added thereto under a nitrogen atmosphere, and the mixture was stirred at room temperature for 1 hour and 15 minutes.

76.1 mg (0.463 mmol) of ammonium pyrrolidine-dithiocarbamate (APDTC) and 152 μL (8.44 mmol) of water were added to the reaction liquid, and the mixture was stirred at room temperature for 45 minutes. The reaction solution was concentrated under reduced pressure, and the residue was dissolved in 18.5 mL of heptane and concentrated again under reduced pressure. The residue was dissolved in 37.0 mL of heptane, cooled to 5° C., and then filtered through Celite. The filtrate was concentrated under reduced pressure, and the residue was dissolved in 86.8 mL of heptane and filtered through a 0.45 μm membrane filter (PTFE type). The filtrate was washed by liquid-liquid extraction with 71.0 mL of acetone, 15.8 mL of water, and 24.0 mL of methanol (MeOH). The upper layer was concentrated under reduced pressure and purified by silica gel column chromatography (heptane/ethyl acetate=6/1→1/3) to afford 1.80 g of Fmoc-Asp(B2-STag)-OH.

ESIMS (m/z) 1147.7 (M+NH₄)⁺

### Reference Example 2

### Synthesis of Fmoc-Glu(B2-STag)-OH

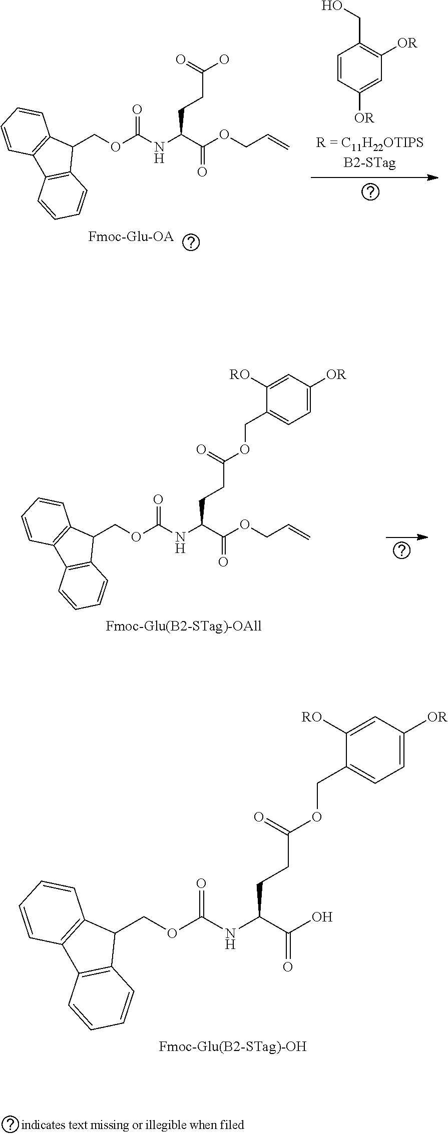

Fmoc-Glu-OAll, Fmoc-Glu(B2-STag)-OAll, and Fmoc-Glu(B2-STag)-OH represent the structures in the formula.

### Reference Example (2-a)

Fmoc-Glu(B2-STag)-OAll was obtained in the same manner as in Reference Example (1-a).

ESIMS (m/z) 1202.0 (M+NH₄)⁺

### Reference Example (2-b)

Fmoc-Glu(B2-STag)-OH was obtained in the same manner as in Reference Example (1-b).

ESIMS (m/z) 1161.8 (M+NH₄)⁺

### Reference Example 3

### Synthesis of Fmoc-Asp(D2-STag)-OH

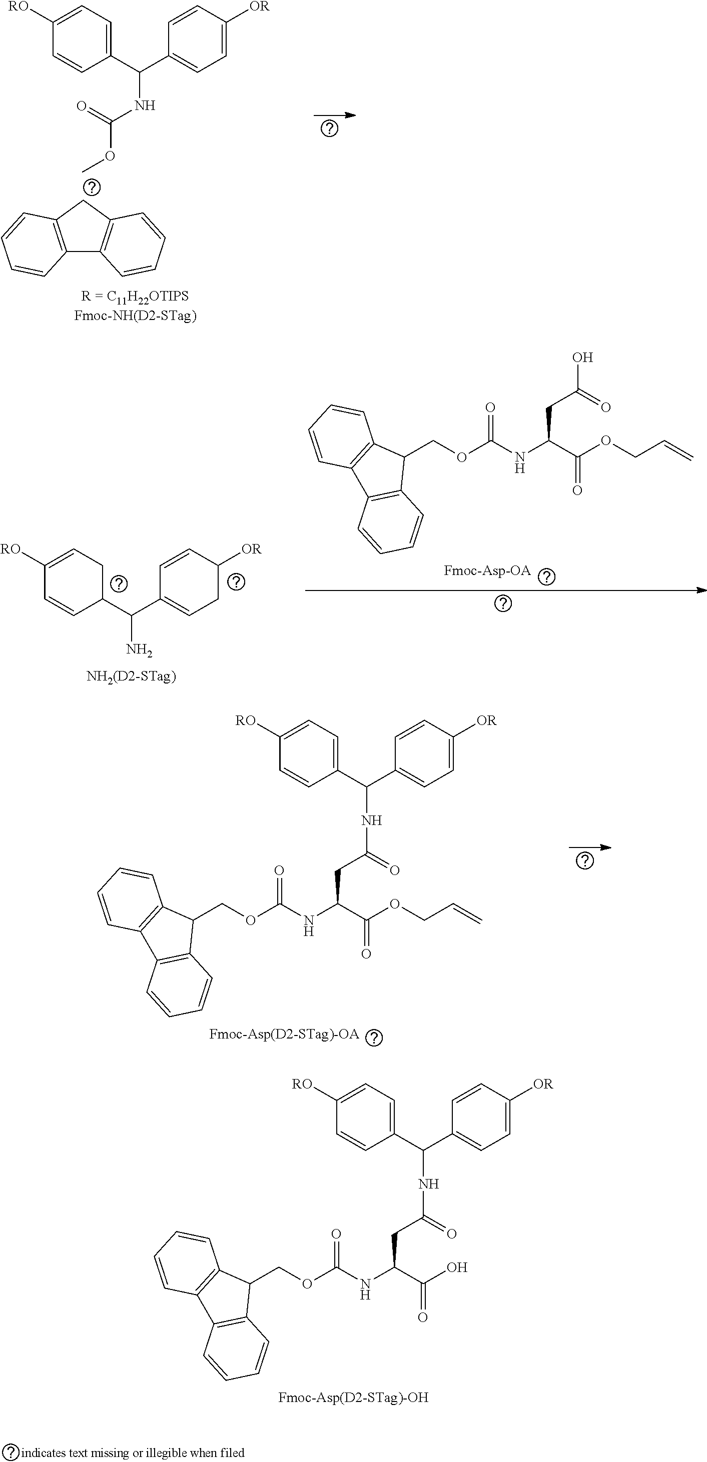

Fmoc-NH(D2-STag), NH2(D2-STag), Fmoc-Asp(D2-STag)-OAll, Fmoc-Asp(D2-STag)-OH represent the structure in the formula.

### Reference Example (3-a)

20.0 g (18.3 mmol) of Fmoc-NH(D2-STag) was dissolved in 293 mL of cyclopentyl methyl ether (CPME), to the mixture were added 73.3 mL of DMF, 5.42 g (30.4 mmol) of sodium 3-mercapto-1-propanesulfonate (MPS) dissolved in 25.5 mL of dimethyl sulfoxide (DMSO), and 1.11 g (6.23 mmol) of solid MPS. Then, thereto was added 5.48 mL (36.7 mmol) of 2,3,4,6,7,8,9,10-octahydropyrimidol[1,2-a]azepine (DBU), and the mixture was stirred at room temperature for 50 minutes. After the mixture was cooled to 5° C., thereto were added 3.19 mL (18.3 mmol) of N,N-diisopropylethylamine (DIPEA), 36.7 mL (36.7 mmol) of 1N sulfuric acid, 241 mL of water, and 10.0 mL of CPME, and the layers were separated. The obtained organic layer was washed by liquid-liquid extraction with 45.5 mL of DMF and 60.7 mL of a 50% aqueous dipotassium hydrogen phosphate solution (50% K₂HPO₄ aqueous solution), to afford 315 mL of a solution mixture containing NH₂ (D2-STag). After 16.8 mL of CPME was added thereto, the solution mixture was divided into two equal parts and used for Reference Example (3-b) and Reference Example (4-a), respectively.

### Reference Example (3-b)

To the solution mixture obtained in Reference Example (3-a), were added 41.5 mL of DMF, 4.35 g (11.0 mmol) of Fmoc-Asp-OAll, 6.39 mL (36.7 mmol) of DIPEA, and 4.71 g (11.0 mmol) of COMU, and the obtained mixture was stirred at room temperature for 30 minutes. The reaction solution was concentrated under reduced pressure. To the residue was added 400 mL of MeOH, and the mixture was stirred, then a solid was collected by filtration.

This washing with MeOH and filtration were further performed twice, and the obtained solid was dried under reduced pressure to afford 9.93 g of Fmoc-Asp(D2-STag)-OAll.

ESIMS (m/z) 1246.0 (M+H)⁺

### Reference Example (3-c)

9.91 g (7.95 mmol) of Fmoc-Asp(D2-STag)-OH was dissolved in 79.5 mL of anhydrous THF, 0.460 g (0.398 mmol) of Pd(Ph₃)₄ and 1.95 mL (15.9 mmol) of phenylsilane were added thereto under a nitrogen atmosphere, and the obtained mixture was stirred at room temperature for 1 hour. 0.448 g (1.99 mmol) of sodium diethyldithiocarbamate (NaDEDTC) and 896 μL (49.8 mmol) of water were added to the reaction liquid, and the mixture was stirred at room temperature for 1 hour. The reaction solution was filtered through a 0.45 μm membrane filter (PTFE type), 15.9 mL of THF and 47.7 mL of DMF were added to the filtrate, and the mixture was concentrated under reduced pressure. The residue was added dropwise to 480 mL of MeOH, and the solid was collected by filtration. The washing with MeOH and the filtration were further performed 2 times, and the obtained solid was dried under reduced pressure to afford 7.93 g of Fmoc-Asp(D2-STag)-OH.

ESIMS (m/z) 1205.8 (M+H)⁺

### Reference Example 4

### Synthesis of Fmoc-Glu(D2-STag)-OH

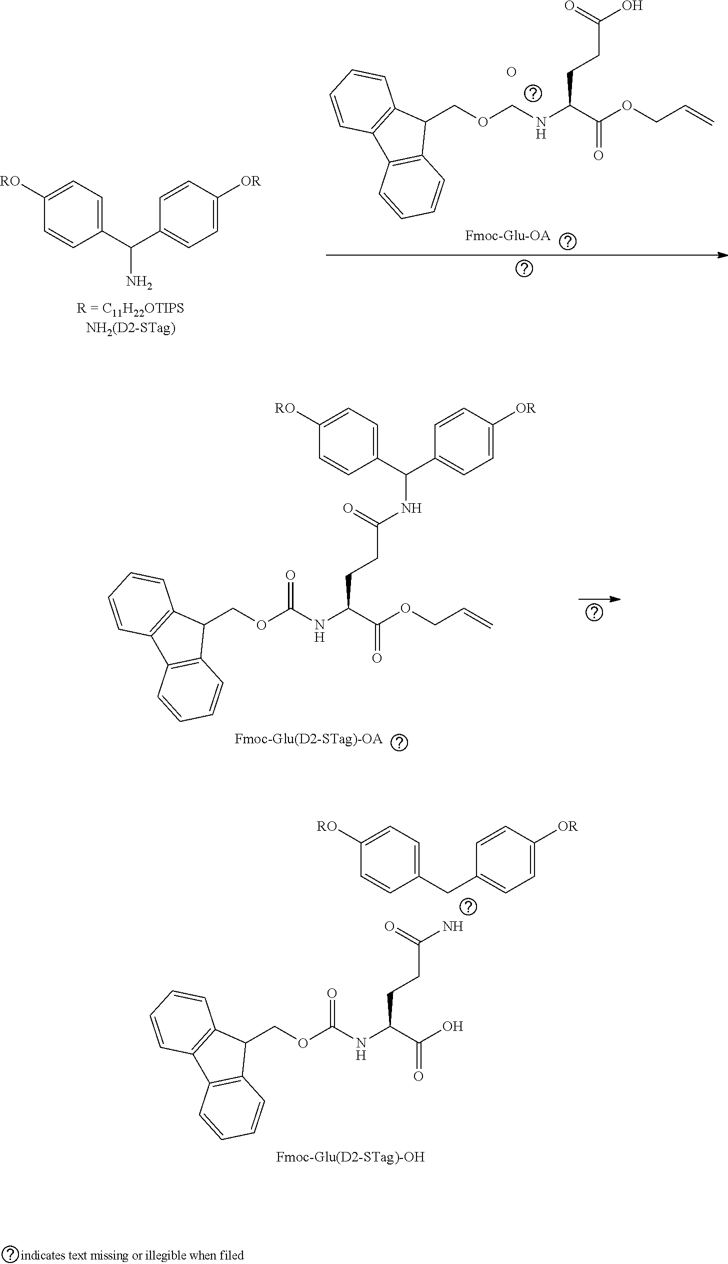

Fmoc-Glu(D2-STag)-OAll and Fmoc-Glu(D2-STag)-OH represent the structure in the formula.

### Reference Example (4-a)

By using the solution mixture obtained in Reference Example (3-a), Fmoc-Glu(D2-STag)-OAll was obtained in the same manner as in Reference Example (3-b).

ESIMS (m/z) 1259.9 (M+H)⁺

### Reference Example (4-b)

Fmoc-Glu(D2-STag)-OH was obtained in the same manner as in Reference Example (3-c).

ESIMS (m/z) 1219.8 (M+H)⁺

When B2-STag is used as a protecting group for the side chains of aspartic acid and glutamic acid, aspartic acid and glutamic acid residues are obtained in the deprotected peptide, respectively. When D2-STag is used as a protecting group for the side chains of aspartic acid and glutamic acid, asparagine and glutamine residues are obtained in the deprotected peptide, respectively. In the peptide synthesis of the present invention, not only aspartic acid and glutamic acid but also peptides containing asparagine and glutamine can be synthesized by appropriately selecting B2-STag and D2-STag as protecting groups of side chains of aspartic acid and glutamic acid.

### Example 1

Synthesis of ⁶⁵⁻⁷⁴ACP using Fmoc-Asp(B2-STag)-OH

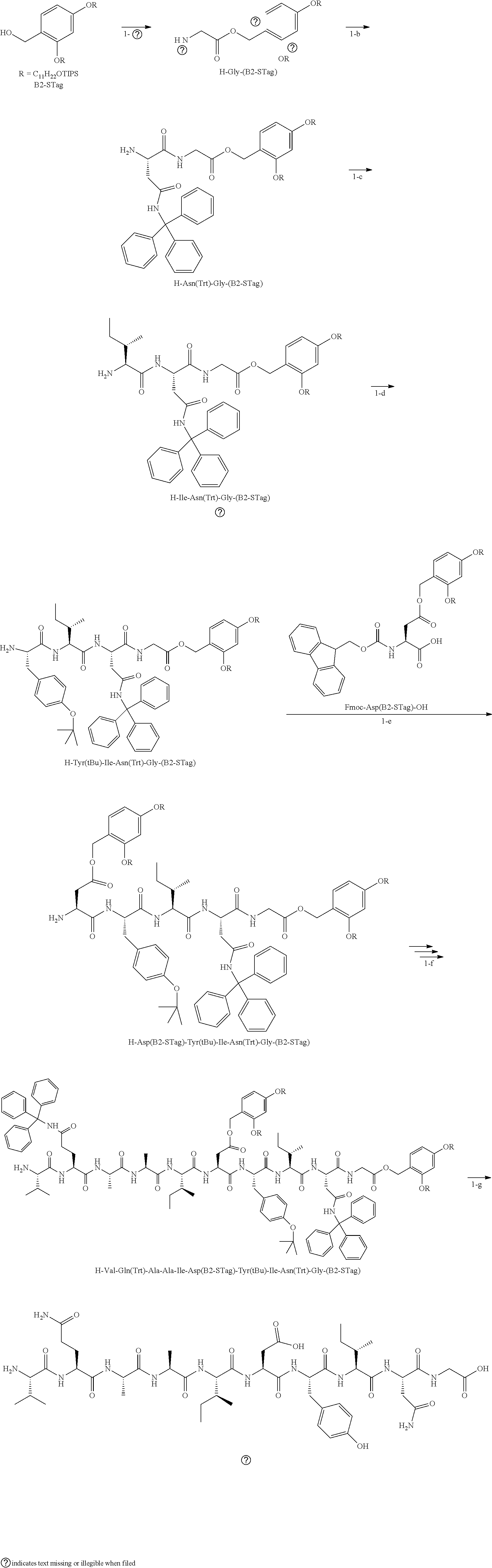

H-Gly-(B2-STag), H-Asn(Trt)-Gly-(B2-STag), H-Ile-Asn(Trt)-Gly-(B2-STag), H-Tyr(tBu)-Ile-Asn(Trt)-Gly-(B2-STag), H-Asp(B2-STag)-Tyr(tBu)-Ile-Asn(Trt)-Gly-(B2-STag), H-Val-Gln(Trt)-Ala-Ala-Ile-Asp(B2-STag)-Tyr(tBu)-Ile-Asn(Trt)-Gly-(B2-STag), and ⁶⁵⁻⁷⁴ACP represents a structure in Formula.

### Example (1-a)

0.500 g (0.630 mmol) of B2-STag was dissolved in 5.00 mL of CPME and 1.25 mL of DMF, and added with 0.562 g (1.89 mmol) of Fmoc-Gly-OH, 0.362 g (1.89 mmol) of EDCI·HCl, and 8.0 mg (0.063 mmol) of DMAP were added and then the mixture was stirred at room temperature for 1 hour. 0.150 mL (1.51 mmol) of 2-(2-aminoethoxy)ethanol (AEE) was added thereto, and the mixture was stirred at room temperature for 15 minutes. 0.322 g (1.81 mmol) of MPS dissolved in 1.51 mL of DMSO and 0.352 g (1.98 mmol) of solid MPS were added, the mixture was cooled to 5° C., 1.22 mL (8.19 mmol) of DBU was added thereto, and the mixture was stirred for 30 minutes. 6.93 mL (6.93 mmol) of 1N sulfuric acid, 4.00 mL of water, and 2.80 mL of CPME were added, and the layers were separated.

The obtained organic layer was washed by liquid-liquid extraction with 1.16 mL of DMF and 1.55 mL of 50% K₂HPO₄ aqueous solution, to afford a solution mixture containing H-Gly-(B2-STag).

### Example (1-b)

0.150 mL of CPME, 2.30 mL of DMF, 0.443 g (0.743 mmol) of Fmoc-Asn(Trt)-OH, 0.431 mL (2.48 mmol) of DIPEA, and 0.318 g (0.743 mmol) of (1-cyano-2-ethoxy-2-oxoethylideneaminooxy)dimethylamino-morpholino-carbenium hexafluorophosphate (COMU) were added to the obtained solution mixture, and the obtained mixture was stirred at room temperature for 40 minutes. 21.0 mg (0.149 mmol) of 2-aminoethyl hydrogen sulfate (2-AEHS) dissolved in 0.594 mL of DMSO was added thereto, and the mixture was stirred at room temperature for 15 minutes. 43.0 mg (0.241 mmol) of MPS dissolved in 0.200 mL of DMSO and 0.222 g (1.25 mmol) of solid MPS were added, the mixture was cooled to 5° C., 0.503 mL (3.37 mmol) of DBU was added thereto, and the mixture was stirred for 1 hour. 3.88 mL (3.88 mmol) of 1N sulfuric acid, 3.80 mL of water, and 0.289 mL of CPME were added, and the layers were separated. The obtained organic layer was washed by liquid-liquid extraction with 1.24 mL of DMF and 1.66 mL of 50% K₂HPO₄ aqueous solution, to afford a solution mixture containing H-Asn(Trt)-Gly-(B2-STag).

### Example (1-c)

0.900 mL of CPME, 2.00 mL of DMF, 0.258 g (0.730 mmol) of Fmoc-Ile-OH, 0.424 mL (2.43 mmol) of DIPEA, and 0.313 g (0.730 mmol) of COMU were added to the obtained solution mixture, and the obtained mixture was stirred at room temperature for 45 minutes. 14.0 IL (0.141 mmol) of 2-(2-aminoethoxy)ethanol (AEE) was added thereto, and the mixture was stirred at room temperature for 15 minutes. 0.166 g (0.931 mmol) of MPS dissolved in 0.781 mL of DMSO and 94.0 mg (0.527 mmol) of solid MPS were added, the mixture was cooled to 5° C., 0.473 mL (3.16 mmol) of DBU was added thereto, and the mixture was stirred for 1 hour. 3.68 mL (3.68 mmol) of 1N sulfuric acid, 4.10 mL of water, and 0.404 mL of CPME were added, and the layers were separated. The obtained organic layer was washed by liquid-liquid extraction with 1.28 mL of DMF and 1.71 mL of 50% K₂HPO₄ aqueous solution, to afford a solution mixture containing H-Ile-Asn(Trt)-Gly-(B2-STag).

### Example (1-d)

In the same manner as in Example (1-c), the peptide was elongated by using Fmoc-Tyr(tBu)-OH to afford 7.80 mL of a solution mixture containing H-Tyr(tBu)-Ile-Asn(Trt)-Gly-(B2-STag). After 0.54 mL of CPME was added to the solution mixture, the mixture was divided into two equal parts, and the half was used for Example (1-e).

### Example (1-e)

1.04 mL of DMF, 0.400 g (0.353 mmol) of Fmoc-Asp(B2-STag)-OH, 0.205 mL (1.18 mmol) of DIPEA, and 0.151 g (0.353 mmol) of COMU were added to the obtained solution mixture, and the obtained mixture was stirred at room temperature for 50 minutes. 10.0 mg (0.0709 mmol) of 2-aminoethyl hydrogen sulfate (2-AEHS) dissolved in 0.283 mL of DMSO was added thereto, and the mixture was stirred at room temperature for 15 minutes. 20.0 mg (0.112 mmol) of MPS dissolved in 95.0 μL of DMSO and 0.106 g (0.595 mmol) of solid MPS were added, the mixture was cooled to 5° C., 0.240 mL (1.60 mmol) of DBU was added thereto, and the mixture was stirred for 30 minutes. 1.85 mL (1.85 mmol) of 1N sulfuric acid, 2.29 mL of water, and 0.249 mL of CPME were added, and the layers were separated. The obtained organic layer was washed by liquid-liquid extraction with 0.660 mL of DMF and 0.880 mL of 50% K₂HPO₄ aqueous solution, to afford a solution mixture containing H-Asp(B2-STag)-Tyr(tBu)-Ile-Asn(Trt)-Gly-(B2-STag). The obtained solution was concentrated and the residue was purified by silica gel column chromatography (heptane/ethyl acetate=1/1) to afford 0.516 g of H-Asp(B2-STag)-Tyr(tBu)-Ile-Asn(Trt)-Gly-(B2-STag).

ESIMS (m/z) 1223.1 (M+H+NH₄)²⁺

### Example (1-f)

In the same manner as in Example (1-b), the peptide was elongated by using Fmoc-Ile-OH, Fmoc-Ala-OH·H₂O, Fmoc-Ala-OH·H₂O, Fmoc-Gln(Trt)-OH, and Fmoc-Val-OH in this order to afford a solution mixture containing H-Val-Gln(Trt)-Ala-Ala-Ile-Asp(B2-STag)-Tyr(tBu)-Ile-Asn(Tr)-Gly-(B2-STag). The solution was concentrated under reduced pressure and to the obtained residue was added 20.0 mL of MeCN/water=9/1. Centrifugation was performed at 5° C. and 4 400 rpm for 1 minute, and the supernatant was removed by decantation to afford a precipitate. 20.0 mL of MeCN was added to the precipitate, the mixture was centrifuged at 4 400 rpm at 5° C. for 1 minute, and the supernatant was removed by decantation to afford a precipitate. This washing with MeCN, centrifugation, and decantation were further performed twice to afford a precipitate. The precipitate was dried under reduced pressure to afford 0.180 g of H-Val-Gln(Trt)-Ala-Ala-Ile-Asp(B2-STag)-Tyr(tBu)-Ile-Asn(Trt)-Gly-(B2-STag).

ESIMS (m/z) 1586.0 (M+H+NH₄)²⁺

### Example (1-g)

A solution mixture of 0.258 mL (3.37 mmol) of trifluoroacetic acid, 6.8 μL (0.0332 mmol) of Triisopropylsilane, and 6.8 μL (0.377 mmol) of water was cooled to 5° C., and 30.0 mg of H-Val-Gln(Trt)-Ala-Ala-Ile-Asp(B2-STag)-Tyr(tBu)-Ile-Asn(Trt)-Gly-(B2-STag) was added. After 3 minutes, the temperature was raised to room temperature, and the mixture was stirred for 3 hours. The reaction liquid was added dropwise to 3.00 mL of methyl tert-butyl ether (MTBE) cooled to 5° C. and centrifuged at 5° C. and 4 400 rpm for 1 minute, and the supernatant was removed by decantation to afford a precipitate. This washing with MTBE, centrifugation, and decantation were further performed three times to afford a precipitate. The precipitate was dried under reduced pressure to afford 7.4 mg of ⁶⁵⁻⁷⁴ACP.

ESIMS (m/z) 1063.8 (M+H)⁺

HPLC Purity: 83.4%

HPLC analysis conditions (1)

Column: Triart C18, 1.9 μm, 12 nm, 150 mm×3.0 mmI.D.

Mobile phase A: 0.1% TFA aqueous solution

Mobile phase B: MeCN containing 0.1% TFA

Flow rate: 0.50 mL/min

Column temperature: 60° C.

Detection wavelength: 225 nm

Gradient conditions: 10% B (0 min)→10% B (5 min) 90% B (25 min)→95% B (25.1 min)→95% B (28.0 min)→10% B (28.1 min)→10% B (35 min)

### Comparative Example 1

Synthesis of H-Asp(tBu)-Tyr(tBu)-Ile-Asn(Trt)-Gly-(B2-STag) using Fmoc-Asp(tBu)-OH

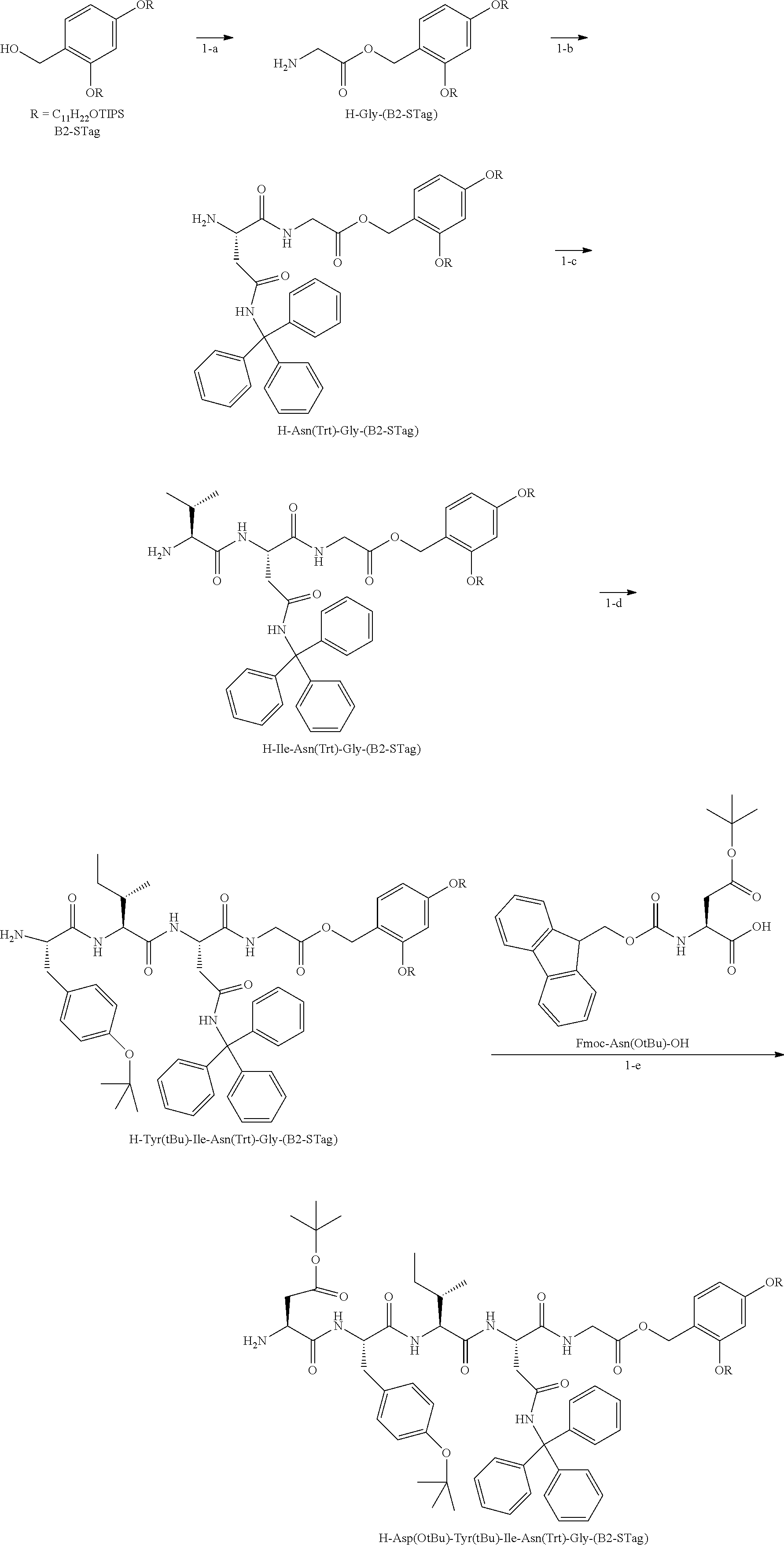

(H-Asp(OtBu)-Tyr(tBu)-Ile-Asn(Trt)-Gly-(B2-STag) denotes the structure in the formula.)

### Comparative Examples (1-a), (1-b), (1-c), and (1-d)

In the same manner as in Examples 1-a, 1-b, 1-c, and 1-d, from 0.800 g (1.01 mmol) of B2-STag, 12.7 mL of a solution mixture containing H-Tyr(tBu)-Ile-Asn(Trt)-Gly-(B2-STag) was obtained.

### Comparative Example (1-e)

1.00 mL of CPME, 3.42 mL of DMF, 0.477 g (1.16 mmol) of Fmoc-Asp(OtBu)-OH, 0.673 mL (3.86 mmol) of DIPEA, and 0.496 g (1.16 mmol) of COMU were added to the obtained solution mixture, and the obtained mixture was stirred at room temperature for 45 minutes. 23.0 μL (0.232 mmol) of AEE was added thereto, and the mixture was stirred at room temperature for 15 minutes. 0.264 g (1.48 mmol) of MPS dissolved in 1.24 mL of DMSO and 0.149 g (0.836 mmol) of solid MPS were added, the mixture was cooled to 5° C. 0.751 mL (5.02 mmol) of DBU was added thereto, and the mixture was stirred for 55 minutes. Subsequently, 5.83 mL (5.83 mmol) of 1N sulfuric acid and 7.40 mL of water were added, and as a result, the organic layer was gelled, and liquid separation operation could not be performed.

ESIMS (m/z) 1710.6 (M+H)⁺

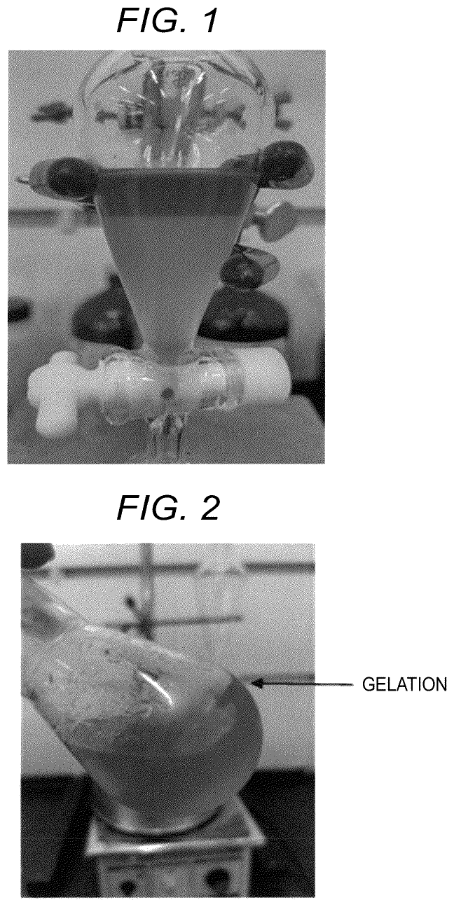

FIGS. 1 and 2 show the state of liquid separation in Example (1-e) and Comparative Example (1-e), respectively. In Example (1-d) shown in FIG. 1, the interface between the organic layer and the aqueous layer was clear, indicating excellent liquid-liquid separation. In contrast, in Comparative Example (1-e) shown in FIG. 2, the organic layer was gelled, which interfered with liquid separation.

Next, the HPLC analysis results of Example 1 and Comparative Example 1 are shown in Table 1. Example 1, where Fmoc-Asp(B2-STag)-OH protected with B2-STag of the present invention was used, showed good liquid separation property, and succeeded in synthesize ⁶⁵⁻⁷⁴ACP with a high purity of 83.4%. In contrast, Comparative Example 1 where Fmoc-Asp(tBu)-OH protected by a tBu group was used instead of B2-STag, prevented liquid separation when H-Asp(tBu)-Tyr(tBu)-Ile-Asn(Trt)-Gly-(B2-STag) was synthesized. In order to continue the synthesis of ⁶⁵⁻⁷⁴ACP, it was necessary to purify H-Asp(tBu)-Tyr(tBu)-Ile-Asn(Trt)-Gly-(B2-STag) by other methods rather than liquid-liquid separation, which was industrially disadvantageous.

TABLE 1
Comparison results of liquid-liquid separation property
between Example 1 and Comparative Example 1

| | Liquid-liquid | |
|---|---|---|
| | separation property *¹ | HPLC purity of ⁶⁵⁻⁷⁴ACP |
| Example 1 | Good | 83.4% |
| Comparative | Impossible to conduct liquid- | Without synthesis |
| Example 1 | liquid separation due to | |
| | gelation | |

*¹ Liquid-liquid separation property in the washing step by liquid-liquid separation with water after quenching with 1N sulfuric acid

From the above results, it was found that by using Fmoc-Asp(B2-STag)-OH in which the side chain of aspartic acid was protected by the B2-STag of the present invention instead of Fmoc-Asp(OtBu)-OH protected by the tBu group, which is a conventional technique, liquid separation property is improved, and a peptide is obtained in an industrially advantageous manner.

### Example 2

### Synthesis of H-Ile-Asp(B2-STag)-Ile-Ala-NH(D2-STag)

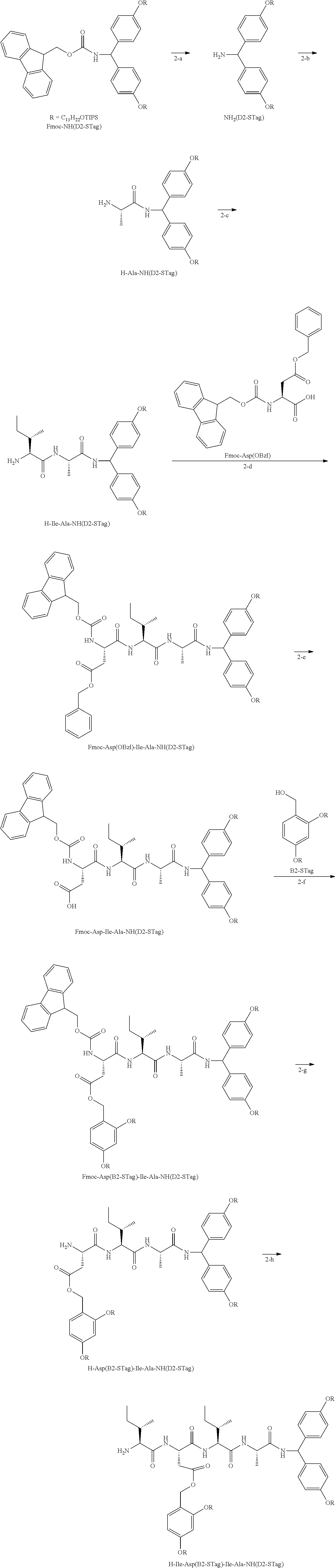

(H-Ala-NH(D2-STag), H-Ile-Ala-NH(D2-STag), Fmoc-Asp(OBzl), Fmoc-Asp(OBzl)-Ile-Ala-NH(D2-STag), Fmoc-Asp-Ile-Ala-NH(D2-STag), Fmoc-Asp(B2-STag)-Ile-Ala-NH(D2-STag), H-Asp(B2-STag)-Ile-Ala-NH(D2-STag), and H-Ile-Asp(B2-STag)-Ile-Ala-NH(D2-STag) represent structures in the formula.)

### Example (2-a)

In the same manner as in Reference Example (3-a), from 5.00 g (4.58 mmol) of Fmoc-NH(D2-STag), 79.0 mL of a solution mixture containing NH₂(D2-STag) was obtained.

### Example (2-b)

CPME (1.20 mL), DMF (38.2 mL), Fmoc-Ala-OH·H₂O (2.26 g (6.88 mmol)), DIPEA (3.19 mL (18.3 mmol)), cyano (hydroxyimino) ethyl acetate (Oxyma) (0.977 g (6.88 mmol)), and COMU (2.94 g (6.88 mmol)) were added to the obtained solution mixture, and the obtained mixture was stirred at room temperature for 40 minutes. 0.273 mL (2.75 mmol) of AEE was added, and the mixture was stirred at room temperature for 15 minutes. 9.80 g (55.0 mmol) of MPS dissolved in 45.8 mL of DMSO and 4.60 mL of DMSO were added thereto, the mixture was cooled to 5° C., 8.22 mL (55.0 mmol) of DBU was added thereto, and the mixture was stirred for 30 minutes. 14.4 mL (57.6 mmol) of 4N hydrochloric acid/CPME, 191 mL of a 20% sodium chloride aqueous solution (20% NaCl aqueous solution), 163 mL of a 10% sodium carbonate aqueous solution (10% Na₂CO₃ aqueous solution), and 1.99 mL of CPME were added, and the layers were separated. 2.90 mL of DMSO, 2.90 mL of DMF, and 106 mL of 50% K₂HPO₄ aqueous solution were added to the obtained organic layer, and the obtained organic layer was washed by liquid-liquid extraction to afford 86.0 mL of a solution mixture containing H-Ala-NH(D2-STag). 19.5 mL of CPME was added to this solution to make 105.0 mL. Among them, 74.0 mL was used for Example (2-c), and 10.5 mL was used for Comparative Example (2-a).

### Example (2-c)

In the same manner as in Example (2-b), the peptide was elongated using Fmoc-Ile-OH to afford 76.5 mL of a solution mixture containing H-Ile-Ala-NH(D2-STag).

ESIMS (m/z) 1052.9 (M+H)⁺

### Example (2-d)

5.6 mL of CPME, 35.2 mL of DMF, 2.14 g (4.81 mmol) of Fmoc-Asp(OBzl)-OH, 2.23 mL (12.8 mmol) of DIPEA, 0.683 g (4.81 mmol) of Oxyma, and 2.06 g (4.81 mmol) of COMU were added to the obtained solution mixture, and the obtained mixture was stirred at room temperature for 30 minutes. The reaction solution was concentrated under reduced pressure, and the residue was added dropwise to 94.9 mL of MeCN/water=9/1, and a solid was collected by filtration. 94.9 mL of MeCN was added to the obtained solid, the mixture was stirred, and the solid was collected by filtration. The washing with MeCN and the filtration were further performed once to afford 3.99 g of a mixture containing Fmoc-Asp(OBzl)-Ile-Ala-NH(D2-STag).

ESIMS (m/z) 1497.0 (M+NH₄)⁺

### Example (2-e)

0.500 g of Fmoc-Asp(OBzl)-Ile-Ala-NH(D2-STag) was dissolved in 3.40 mL of THF, 0.111 g of Pd/C (STD type, Pd 5%, (hydrous)) was added thereto, and the mixture was stirred at 4:30 at room temperature under a hydrogen atmosphere. The reaction liquid was filtered through Celite, and the filtrate was concentrated and dried under reduced pressure to afford 0.463 g of a mixture containing Fmoc-Asp-Ile-Ala-NH(D2-STag).

ESIMS (m/z) 1389.5 (M+H)⁺

### Example (2-f)

0.410 g of Fmoc-Asp-Ile-Ala-NH(D2-STag) and 0.468 g (0.590 mmol) of B2-STag were dissolved in a solution mixture of 4.13 mL of CPME and 1.77 mL of DMF, 28.8 mg (0.236 mmol) of DMAP and 62.2 mg (0.324 mmol) of EDCI·HCl were added, and the mixture was stirred at room temperature for 5 hours. Further, 6.2 mg (0.0323 mmol) of EDCI·HCl was added thereto, and the obtained mixture was stirred at room temperature for 1 hour. The reaction liquid was concentrated, 25.6 mL of MeCN was then added to the residue, and the supernatant was removed by decantation to afford a precipitate. This washing with MeCN and decantation were additionally performed 2 times, and the obtained precipitate was dried under reduced pressure to afford 0.707 g of a mixture containing Fmoc-Asp(B2-STag)-Ile-Ala-NH(D2-STag).

ESIMS (m/z) 1099.6 (M+2NH₄)²⁺

### Example (2-g)

0.639 g of Fmoc-Asp(B2-STag)-Ile-Ala-NH(D2-STag) was dissolved in a solution mixture of 4.70 mL of CPME and 1.20 mL of DMF, and added with 87.0 mg (0.488 mmol) of MPS dissolved in 0.410 mL of DMSO, 18.0 mg (0.101 mmol) of solid MPS, and 0.0882 mL (0.590 mmol) of DBU, and the mixture were stirred at room temperature for 40 minutes. The reaction solution was cooled to 5° C., 51.4 μL (0.295 mmol) of DIPEA was added thereto, 0.590 mL (0.590 mmol) of 1N sulfuric acid, 4.00 mL of water, and 0.319 mL of CPME were added thereto, and the layers were separated. 0.800 mL of DMF and 1.00 mL of 50% K₂HPO₄ aqueous solution were added to the obtained organic layer, and the obtained organic layer was washed by liquid-liquid extraction. The obtained upper layer was concentrated under reduced pressure, and the residue was purified by silica gel column chromatography (heptane/ethyl acetate=5/1→0/100) to afford 0.329 g of H-Asp(B2-STag)-Ile-Ala-NH(D2-STag).

ESIMS (m/z) 1942.6 (M+H)⁺

### Example (2-h)

0.329 g (0.169 mmol) of H-Asp(B2-STag)-Ile-Ala-NH(D2-STag) was dissolved in a solution mixture of 4.50 mL of CPME and 1.90 mL of DMF, and added with 0.0898 g (0.254 mmol) of Fmoc-Ile-OH, 0.110 mL (0.678 mmol) of DIPEA, 0.0361 g (0.254 mmol) of Oxyma, and 0.109 g (0.254 mmol) of COMU, and the mixture was stirred at room temperature for 50 minutes. 10.0 μL (0.101 mmol) of AEE was added thereto, and the mixture was stirred at room temperature for 15 minutes.

0.362 g (2.03 mmol) of MPS dissolved in 1.69 mL of DMSO and 0.169 mL of DMSO were added thereto, the mixture was cooled to 5° C., 0.203 mL (1.36 mmol) of DBU was added thereto, and the mixture was stirred for 30 minutes. 0.356 mL (1.42 mmol) of 4N hydrochloric acid/CPME, 8.10 mL of 20% NaCl aqueous solution, 7.00 mL of 10% Na₂CO₃ aqueous solution, and 0.165 mL of CPME were added, and the layers were separated. 0.135 mL of DMSO, 0.135 mL of DMF, and 5.00 mL of 50% K₂HPO₄ aqueous solution were added to the obtained organic layer, and the obtained organic layer was washed by liquid-liquid extraction. The obtained upper layer was concentrated under reduced pressure, 7.00 mL of MeCN/water=9/1 was added to the residue, and the supernatant was removed by decantation to afford a precipitate. 7.00 mL of MeCN was added to the precipitate, and the supernatant was removed by decantation to afford a precipitate. This washing with MeCN and decantation were further performed once to afford a precipitate. The obtained precipitate was dried under reduced pressure to afford 0.293 g of a mixture containing H-Ile-Asp(B2-STag)-Ile-Ala-NH(D2-STag).

ESIMS (m/z) 1037.2 (M+H+NH₄)²⁺

### Comparative Example 2

### Synthesis of H-Ile-Asp(tBu)-Ile-Ala-NH(D2-STag)

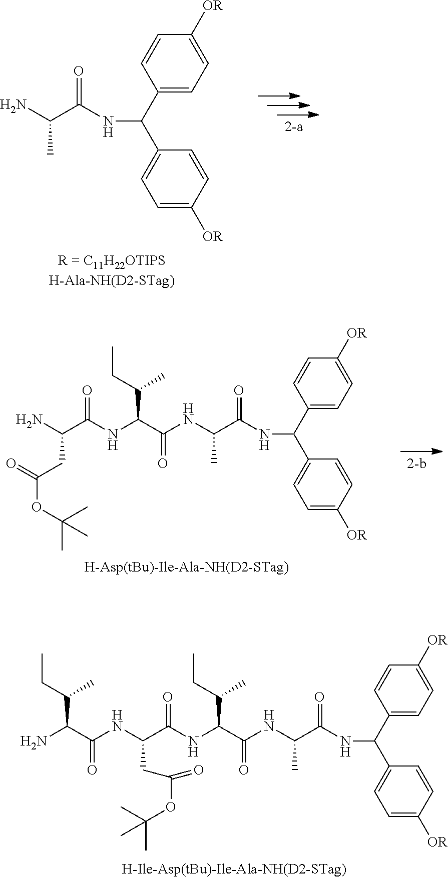

H-Asp(tBu)-Ile-Ala-NH (D2-STag) and H-Ile-Asp(tBu)-Ile-Ala-NH(D2-STag) represent structures in the formula.

### Comparative Example (2-a)

Using 10.5 mL of the solution mixture containing H-Ala-NH(D2-STag) obtained in Example (2-b), the peptide was elongated using Fmoc-Ile-OH and Fmoc-Asp(tBu)-OH in the same manner as in Example (2-b) to afford 11.3 mL of a mixed liquid containing H-Asp(tBu)-Ile-Ala-NH (D2-STag).

ESIMS (m/z) 1224.0 (M+H)⁺

### Comparative Example (2-b)

0.700 mL of CPME, 5.10 mL of DMF, 0.240 g (0.680 mmol) of Fmoc-Ile-OH, 0.316 mL (1.81 mmol) of DIPEA, 0.0967 g (0.680 mmol) of Oxyma, and 0.291 g (0.680 mmol) of COMU were added to the obtained solution mixture, and the obtained mixture was stirred at room temperature for 50 minutes. Incidentally, a gel-like insoluble matter was precipitated during the condensation reaction. 27.0 μL (0.272 mmol) of AEE was added thereto, and the mixture was stirred at room temperature for 15 minutes. 0.970 g (5.44 mmol) of MPS dissolved in 4.53 mL of DMSO and 0.453 mL of DMSO were added, the mixture was cooled to 5° C., 0.542 mL (3.63 mmol) of DBU was added thereto, and the mixture was stirred for 1 hour. Since the reaction was not completed, the temperature was raised to room temperature, and the mixture was further stirred for 1 hour. Incidentally, gel-like insoluble matters were precipitated even during this deFmoc reaction. 0.952 mL (3.81 mmol) of 4N hydrochloric acid/CPME, 21.8 mL of 20% NaCl aqueous solution, 18.7 mL of 10% Na₂CO₃ aqueous solution, and 0.277 mL of CPME were added, and the layers were separated. As 0.357 mL of DMSO, 0.357 mL of DMF, and 13.2 mL of 50% K₂HPO₄ aqueous solution were added to the obtained organic layer, the solution was gelled, and therefore the liquid separation operation was not to be performed. The obtained gel-like material was concentrated under reduced pressure. To the residue was added 24.2 mL of MeCN/water=9/1, and a solid was collected by filtration. To the solid was added 12.1 mL of MeCN, and the solid was collected by filtration. The washing with MeCN and the filtration were further performed once, and the afforded solid was dried under reduced pressure to afford 0.339 g of a mixture containing H-Ile-Asp(tBu)-Ile-Ala-NH (D2-STag).

ESIMS (m/z) 1337.2 (M+H)⁺

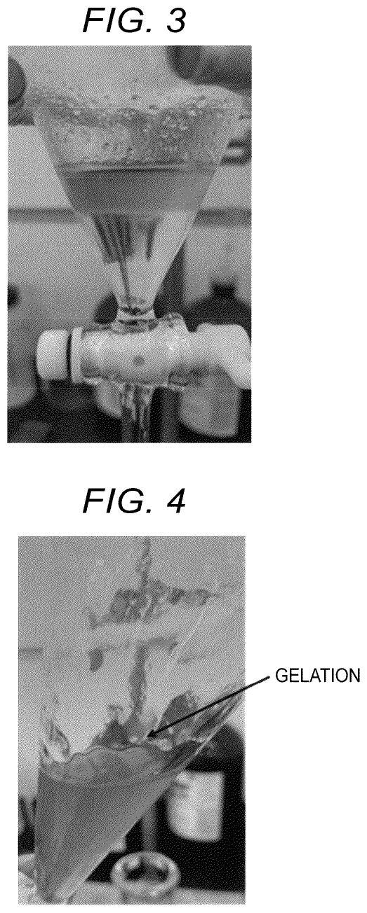

FIGS. 3 and 4 show the state of liquid separation in Example (2-h) and Comparative Example (2-b). In Example (2-h) shown in FIG. 3, the interface between the organic layer and the aqueous layer was clear, with good liquid-liquid separation. In contrast, in Comparative Example (2-b) shown in FIG. 4, the organic phase was gelled, and the liquid separation operation could not be performed.

Next, synthesis results of Example (2-h) and Comparative Example (2-b) are shown in Table 2. In Example (2-h) in which the side chain of aspartic acid was protected by B2-STag of the present invention, the deFmoc reaction progressed in a fast rate, with good liquid separation property. In contrast, in Comparative Example (2-b) in which the side chain of asparagine was protected by a tBu group instead of B2-STag, the reaction rate of the deFmoc reaction was slow, and liquid separation was not possible. Therefore, Comparative Example (2-b) was industrially disadvantageous in terms of reactivity and operability as compared with Example (2-h).

TABLE 2
Comparison of synthesis results of Example
(2-h) and Comparative Example (2-b)

| | | Liquid-liquid |
|---|---|---|
| | Conversion rate of deFmoc | separation |
| | reaction *² | property *³ |
| Example | Reaction temperature 5° C., | Good |
| (2-h) | reaction elapsed time 8 min: 99.8%. | |
| Comparative | Reaction temperature 5° C., | Liquid- |
| Example | reaction elapsed time 8 min: 61.6%. | liquid |
| (2-b) | Reaction temperature 5° C., | separation |
| | reaction elapsed time 43 min: | is not |
| | 77.6% (At 1 h, the reaction | possible |
| | temperature is raised from 5° C. to | |
| | room temperature.) | |
| | Room temperature, reaction | |
| | elapsed time 1 h 17 min: 98.7% | |
| | Room temperature, reaction | |
| | elapsed time 1 h 50 min: 99.8% | |

*² The Area % of raw material and target product is measured by HPLC and calculated by the following formula. Conversion rate (%) = (Area % of target product/(Areas of raw material + Area % of target product)) × 100
*³ Liquid-liquid separation property in the washing step by liquid-liquid separation with DMSO, DMF, and 50% K₂HPO₄ aqueous solution

HPLC analysis conditions (2)

Column: YMC Pack Pro C18 250×4.6 mm, S-5 μm, 12 nm

Mobile phase A: 500 mM aqueous sodium perchlorate solution

Mobile phase B: THF

Flow rate: 1.0 mL/min

Column temperature: 45° C.

Detection wavelength: 280 nm

Gradient conditions: 75% B (0 min)→90% B (10 min)→90% B (20 min)→75% B (21 min)→75% B (33 min)

From the above results, it was found that protecting the side chain of aspartic acid with B2-STag instead of the tBu group served to improve reactivity and liquid separation property, to produce a peptide in an industrially advantageous manner.

1. A method for synthesizing a peptide comprising one or more amino acid having a functional group in a side chain thereof, 50% or more of hydrophobic amino acid, and 10% or less of amino acid in which an α-amino group is a secondary amino group; or a peptide comprising one or more amino acid having a functional group in a side chain thereof, and 21 or more of amino acid residues,
wherein a carrier for peptide synthesis represented by a general Formula (1) below is introduced into the side chain of the amino acid having a functional group in a side chain thereof: 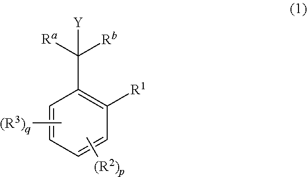
wherein,
R¹ is a hydrogen atom; or when R^{b} is a group represented by Formula (a) below, R¹ is a fluorene ring taken together with Formula (a), a xanthene ring taken together with Formula (a) via an oxygen atom, or a 9,10-dihydroanthracene ring taken together with Formula (a) via a methylene group;
p R²'s each independently represent a group represented by Formula (2); 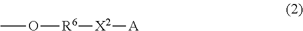
R⁶ represents a linear or branched alkylene group having 1 to 16 carbon atoms;
X² represents O or CONR¹⁷ (wherein R¹⁷ represents a hydrogen atom or an alkyl group having 1 to 4 carbon atoms); and
A represents a group represented by Formula (3), (4), (5), (6), (7), (8), (9), (10), (11), (12) or (13), 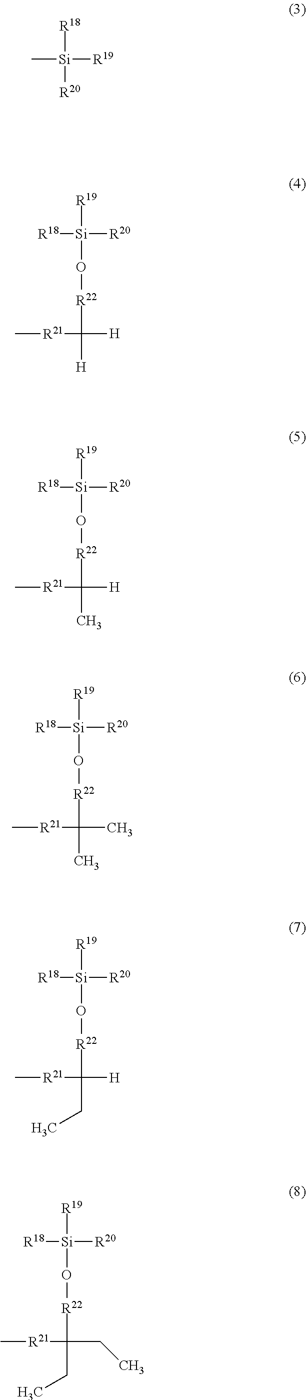 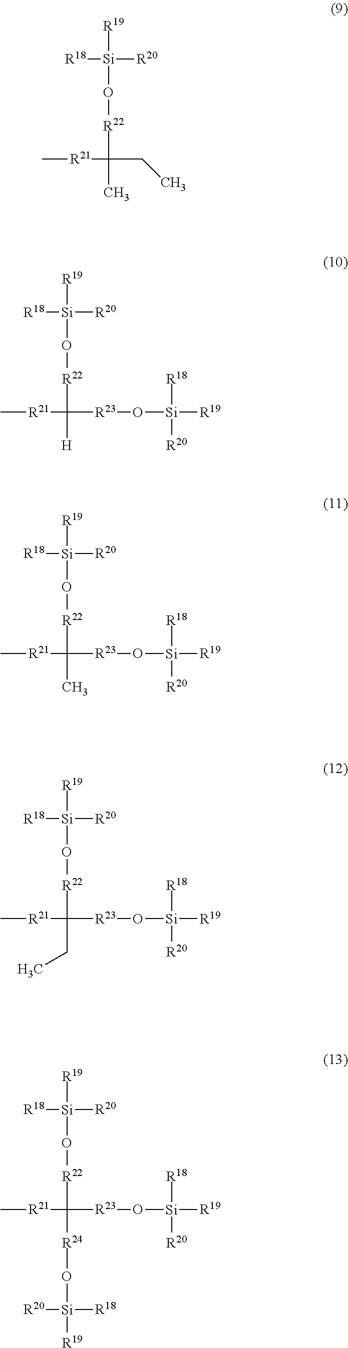
wherein, R¹⁸, R¹⁹, and R²⁰ are the same or different and each represent a linear or branched alkyl group having 1 to 6 carbon atoms or an aryl group optionally having a substituent; and R²¹ represents a single bond or a linear or branched alkylene group having 1 to 3 carbon atoms, and R²², R²³, and R²⁴ each represent a linear or branched alkylene group having 1 to 3 carbon atoms,
q R³'s each independently represent a hydrogen atom, a halogen atom, an alkyl group having 1 to 4 carbon atoms, or an alkoxy group having 1 to 4 carbon atoms;
p represents an integer of 1 to 4, and q represents an integer of 0 to 3; and
R^{a} and R^{b} each independently represent a hydrogen atom, a linear or branched alkyl group having 1 to 4 carbon atoms, an aralkyl group, or a group represented by Formula (a); 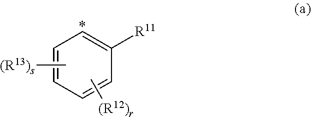
wherein, * represents a bonding site;
R¹¹ may be a hydrogen atom; or may form a fluorene ring taken together with R¹ in Formula (1), a xanthene ring taken together with R¹ in Formula (1) via an oxygen atom, or a 9,10-dihydroanthracene ring taken together with R¹ in Formula (1) via a methylene group;
r R¹²'s each independently represent a group represented by Formula (2) above (R⁶, X², and A in Formula (2) are the same as described above);
s R¹³'s each independently represent a hydrogen atom, a halogen atom, an alkyl group having 1 to 4 carbon atoms, or an alkoxy group having 1 to 4 carbon atoms; and
s represents an integer of 1 to 4 and r represents an integer of 0 to 3, and Y represents a hydroxy group, NHR²⁵ (R²⁵ represents a hydrogen atom, an alkyl group, or an aralkyl group), or a halogen atom.

2. The method for synthesizing a peptide according to claim 1, wherein the hydrophobic amino acid is selected from the group consisting of glycine, alanine, valine, leucine, isoleucine, methionine, phenylalanine, and tryptophan.

3. The method for synthesizing a peptide according to claim 1, wherein a number of amino acid in which the carrier for peptide synthesis represented by the general Formula (1) is introduced into the side chain is 1 to 4 residues.

4. The method for synthesizing a peptide according to claim 1, wherein the amino acid having a functional group in a side chain thereof is an amino acid in which the carrier for peptide synthesis represented by the general Formula (1) is previously introduced into the side chain.

5. The method for synthesizing a peptide according to claim 1, wherein the carrier for peptide synthesis represented by the general Formula (1) is introduced after an amino acid side chain contained in a peptide is deprotected.

6. The method for synthesizing a peptide according to claim 1, wherein the amino acid in which the carrier for peptide synthesis represented by the general Formula (1) is introduced into the side chain is selected from the group consisting of aspartic acid, glutamic acid, and cysteine.

7. The method for synthesizing a peptide according to claim 1, which is a liquid-phase synthesis method.
